(19) United States
(12) Patent Application Publication
(10) Pub. No.: US 2008/0319440 A1
(43) Pub. Date: Dec. 25, 2008

(54) ENDOSCOPIC UNIVERSAL HANDLE

(76) Inventors: Theodore A. Richardson, Cincinnati, OH (US); James David Hughett, SR., Liberty Township, OH (US)

Correspondence Address: COOK ALEX LTD, SUITE 2850, 200 WEST ADAMS STREET, CHICAGO, IL, 60606, US

(21) Appl. No.: 11/764,850

(22) Filed: Jun. 19, 2007

(51) Int. Cl. A61B 18/14

(52) U.S. Cl. 606/42

(57) ABSTRACT

An electrosurgical system is provided having a handpiece adapted to receive a plurality of different end effectors such that, when a particular end effector is attached to the handpiece, the end effector is recognized by either or both the handpiece and a separate electrosurgical generator so that the operational parameters for the particular end effector attached to the handpiece are selected.

### FIELD OF THE INVENTION

The present invention relates, in general, to electrosurgical systems and devices and, more particularly, to bipolar electrosurgical systems with interchangeable tools.

### BACKGROUND OF THE INVENTION

The present invention relates to surgical instruments and systems, with some embodiments relating to different electrosurgical devices such as clamps, articulated clamps, and tissue ablating clamps. Surgery generally refers to the diagnosis or treatment of injury, deformity, or disease. In a variety of surgical procedures, it is desired to ablate tissue or cause lesions in tissue. Some examples of such procedures include, without limitation, electrical isolation of the pulmonary veins to treat atrial fibrillation, ablation of uterine tissue associated with endometriosis, ablation of esophageal tissue associated with Barrett's esophagus, ablation of cancerous liver tissue, and the like. The foregoing examples are merely illustrative and not exhaustive.

Depending on the surgeon's needs, different electrosurgical devices can be plugged into and used with the same electrosurgical generator to form an electrosurgical system with different energy applying capabilities. While a variety of techniques and devices have been used to ablate or cause lesions in tissue, no one has previously made or used an electrosurgical system and electrosurgical instruments in accordance with the present invention. Other aspects of the present teaching relate to non-energy based surgical instruments and are not limited to tissue ablation.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 is a perspective view of a universal handpiece according to the present invention with a separate shaft assembly.

FIG. 2 is a fragmentary side view of the handpiece of FIG. 1.

FIG. 3 is a cross-sectional view of the handpiece taken along line 2-2 of FIG. 2.

FIG. 4 is a cross sectional view of the handpiece taken along line 4-4 of FIG. 3.

FIGS. 5 and 6 are cross-sectional views taken along line 5-5 of FIG. 2 with the shaft retention lock in its shaft engaging and shaft releasing positions, respectively.

### DETAILED DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

In a variety of surgical procedures, it is desirable to ablate tissue or cause lesions in tissue. Tissue ablation can be effected through a variety of different mechanisms known to those skilled in the art, such as mono-polar radiofrequency (“RF”) energy, bi-polar RF energy, cryogenic techniques, and the like. In clamping arrangements, tissue ablation can be effected through a single jaw of a clamp or through both jaws of a clamp. Tissue ablation will typically be performed once the target tissue is clamped between the closed jaws. One with ordinary skill in the art will recognize that one or more of the foregoing tissue ablation techniques may be employed with the various clamp configurations described below. One with ordinary skill in the art will also recognize advantages of the surgical clamps without tissue ablation functionality. Accordingly, the foregoing examples may or may not include ablation functionality. Examples of tissue ablating clamps are found in U.S. patent application Ser. No. 11/254,075, filed Oct. 19, 2005, which is incorporated by reference herein in its entirety.

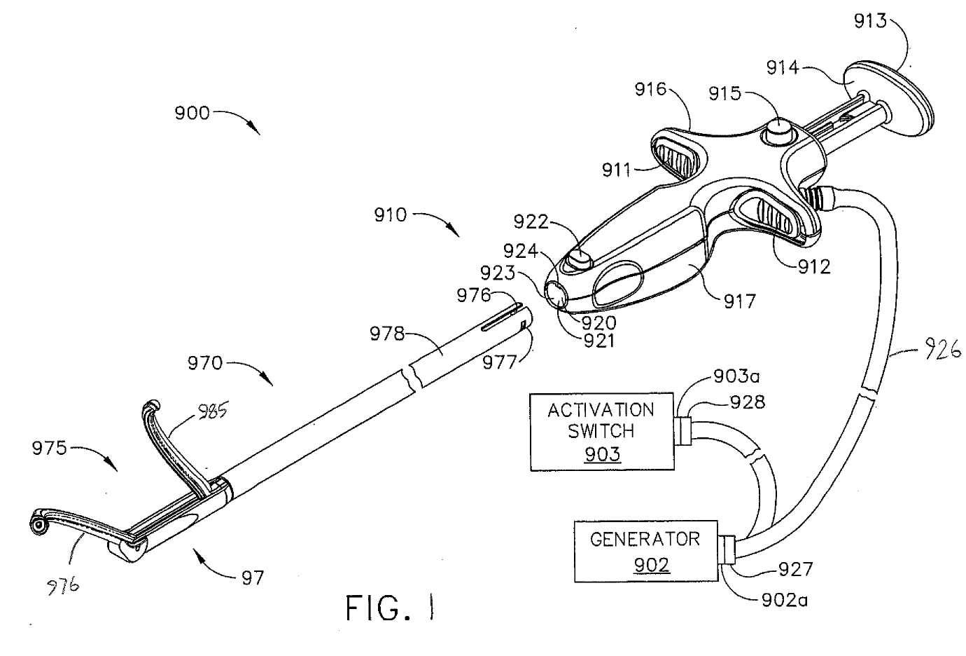

As shown in FIG. 1 electrosurgical instruments have an electrosurgical generator 902 to power the electrosurgical instrument. Electrosurgical generator 902 has a connection port 902a into which a variety of electrosurgical devices or tools (such as clamps described above) can be operably attached and detached. Electrosurgical generator 902 may support ablation, impedance sensing, provide preprogrammed cauterization algorithms to adjust energy settings, or any one of a number of other feature, and can accommodate a wide range of electrosurgical instruments. These electrosurgical instruments may include but are not limited to LED's, sensors, thermocouples, dissectors, energy delivery devices, fasteners, cutters, clamps, actuators and the like, and each combination can have different power needs or require different energy delivery algorithms. Electrosurgical generator 902 is flexible enough to support this wide variety of electrosurgical needs, but different generator settings may be required when shifting from one electrosurgical device to another. A switch or foot pedal 903 having connector 903a can be provided to actuate an electrosurgical tool.

Alternately, instead of a number of electrosurgical devices, a universal handpiece or universal handle 910 can be provided that can accommodate a plurality of interchangeable shaft assemblies 970. The interchangeable shaft assemblies 970, in combination with universal handle 910, can be assembled, disassembled and reassembled into a variety of electrosurgical devices including the clamps described above. Additionally, the universal handle 910 may include an operator actuatable trigger or plunger 914 to actuate mechanical elements of a shaft assembly 910. The combination of universal handle 910 and shaft assemblies 970 could contain electronic elements that provide the electrosurgical generator 902 with information as to which tool or shaft assembly 970 is attached. With this information, generator 902 could perform any one of a number of functions that can include, but is not restricted to, activating sensors, actuating LED's and adjusting power algorithms and voltages automatically without surgeon intervention. A number of interchangeable shaft assemblies such as shaft assembly 970 may be provided for the universal handle 910. Each of the shaft assemblies 970 can have a unique distal end effector 975 that performs a set of functions. By way of example, but not inclusive, a left clamp assembly, a dissector shaft, a right clamp assembly or a shaft assembly (none of which are shown) could be attached to universal handle 910. It would be obvious to one skilled in the art that a number of alternate interchangeable shaft assemblies 970 with different end effectors 975 could be constructed. Additionally, one with ordinary skill in the art will also recognize advantages of the shaft assemblies without tissue ablation functionality that can be used with an electrosurgical system. Accordingly, the foregoing examples may or may not include ablation functionality

### Endoscopic Universal Handle

In FIG. 1, an interchangeable shaft assembly 970 is shown positioned for insertion into a shaft socket 920 at a distal end of universal handle 910. Shaft socket 920 may include a plurality of internal electrical pins 921, a shaft retention lock 922, a drive member 923, and an alignment key 924. Placement of interchangeable shaft assembly 970 into shaft socket 920 aligns shaft 970 with key 924 by means of slot 976, engages internal electrical pins 921 with shaft 970, detachably secures shaft 970 to handle 910 with shaft retention lock 922, and rigidly connects or engages drive member 923 with shaft 970 for actuation of an end effector 975.

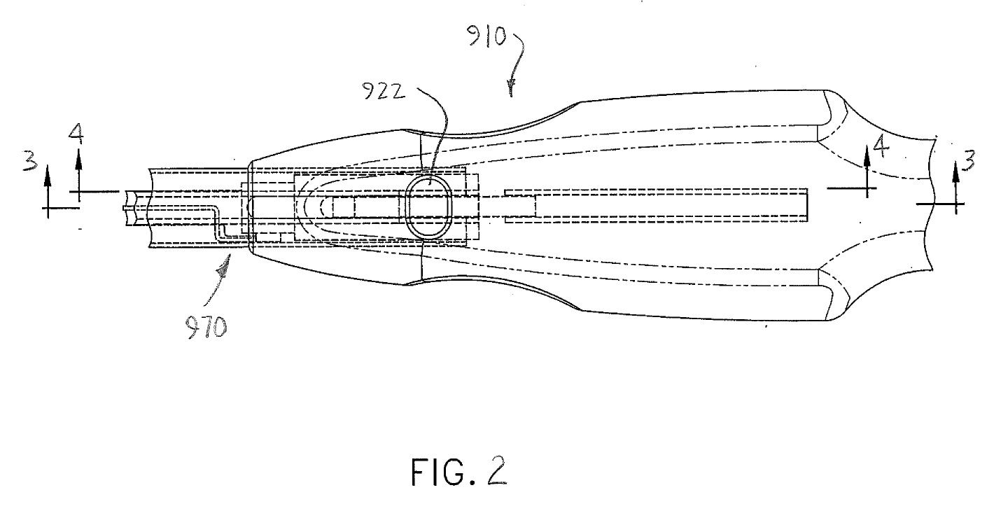

Universal handle 910 has grips 911, 912, 913 and a plunger 914. A plunger 914 is operably connected to internal drive member 923 such that activation or depression of plunger 914 may actuate end effectors 975 in a number of interchangeable shaft assemblies 970. That is, longitudinal motion and forces applied to plunger 914 can be transmitted to internal drive member 923 and then to shaft actuation mechanisms such as end effector 975 in universal shaft 910. Any one of a number of linear motion drive mechanisms, return springs, and end effector force limiting mechanisms as are known in the art can be used in any combination in universal handle 910. A lock 915 holds depressed plunger 914 at different actuation positions when plunger 914 moves distally to actuate end effectors 975. Depressing lock 915 releases a depressed and locked plunger 914 to return drive member 923 proximally and sequentially to at least one intermediate position and then to un-actuated position of FIGS. 1-2. In addition to foot pedal 903, a generator activation switch 916 may be provided on universal handle 910 adjacent to the grips 911, 912, 913 to energize electrosurgical generator 902. A cover 917 can enclose internal handle mechanical elements. Wires 926 can extend from handle 910 and can be terminated with a power connector 927 and an activation connector 928.

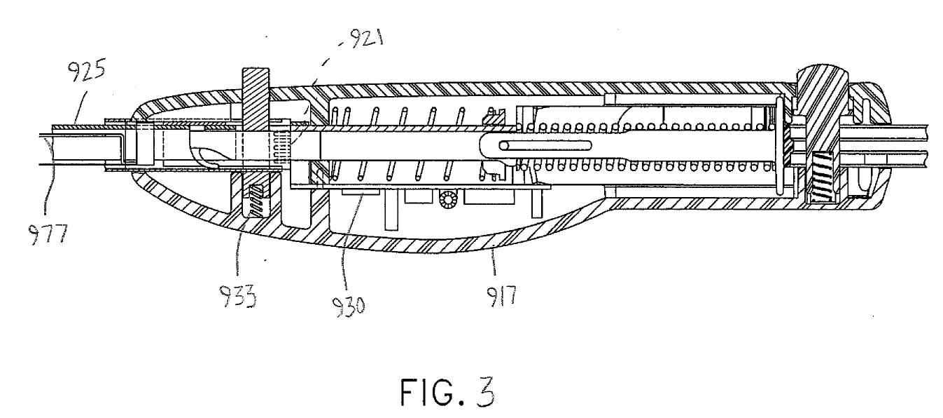

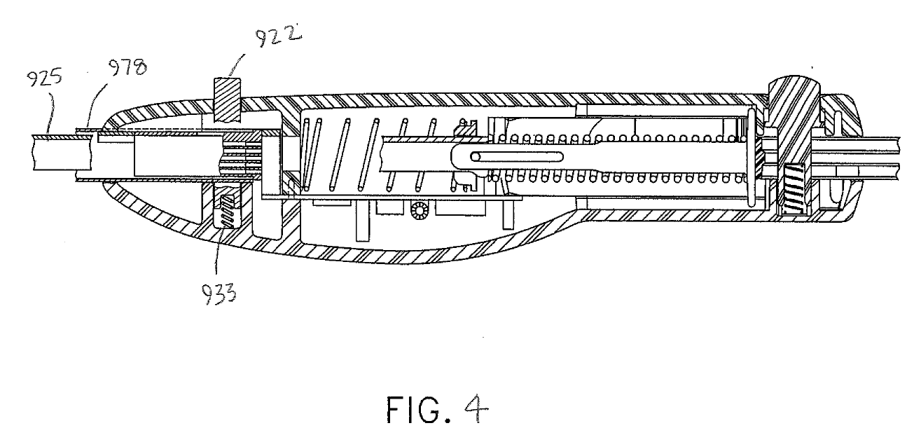

FIGS. 3-4 show a cross sectional view of the universal handle 910 and an interchangeable shaft assembly 970 having end effector 975. Shaft assembly 970 is a non-limiting example, meant to merely illustrate the engagements, interconnections, and operations of universal handle 910 with an example shaft assembly 970 and an example end effector 975. A circuit board 930 is located in a bottom portion of cover 917 below actuation mechanism 925 and an insulator 931 is provided between actuation mechanism 925 and circuit board 930 to prevent shorting. A connector having a plurality of electrical pins 921 extending distally therefrom electrically connects the circuit board 930 with shaft assembly 970. Pins 921 can extend through an opening in shaft retention lock 922. A central opening is located within connector pins 921, and allows longitudinal passage of internal drive member 923 of actuation mechanism 925 therethrough. Shaft retention lock 922 is normally biased by spring 933 to the lock position shown.

### Shaft Assembly

Referring to FIG. 1, the shaft assembly 970 shows a clamp-type end effector including a longitudinally moveable proximal jaw 985 and an articulating distal jaw 976. A longitudinally moveable actuator shaft or drive member 977 extends proximally from proximal jaw 985 in a shaft 980. A female connector 978 is located at a proximal end of shaft 980 and contains a plurality of female sockets 979 to engage with pins 921 of handpiece 910. An opening extends through female connector 978 and has a proximal end of the actuator shaft 977 longitudinally movable therein. Slot 976 extends longitudinally in connector 978 and shaft 980 to receive alignment key 924 of handle 910 therein, and allow passage of an unlocking blade 922b of shaft retention lock 922 therethrough. Electrical elements such as but not limited to an EEPROM or shaft electronics 981 and an electrode wire 982 are shown electrically connected to female connector 978.

FIGS. 3-6 show the attachment of an illustrative interchangeable shaft assembly 970 to the universal handpiece 910. FIG. 3 is a cross sectional view of a portion of the proximal end of handle 910 and interchangeable shaft assembly 970. Shaft 970 is rotated into alignment with alignment key 924 (allowing key 924 to slide in slot 976) and is shown partially inserted into shaft socket 920. Pins 921 of handle 910 are aligned with female sockets 979 of handle 910 to ensure proper connection.

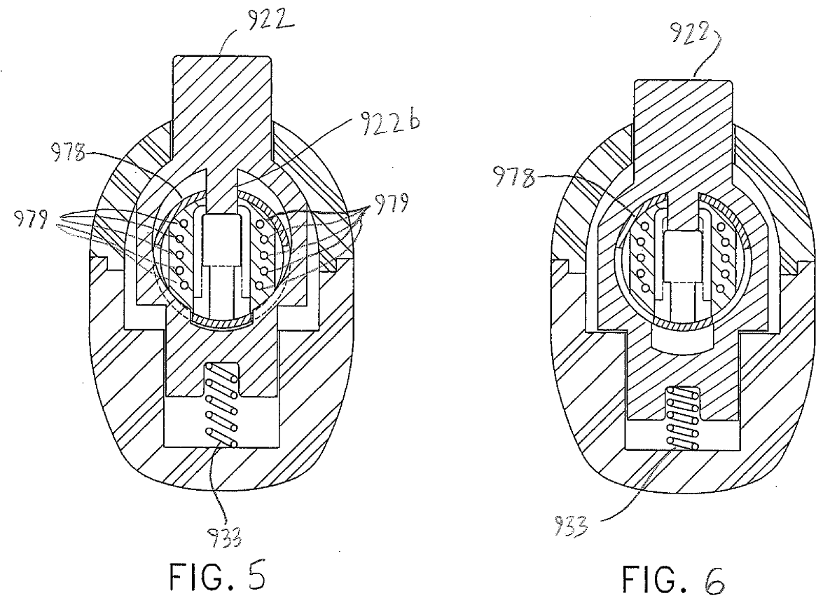

FIGS. 5 and 6 are section views of handle 910 and show an end view of interchangeable shaft assembly 910 viewed through opening 922a of shaft release button 922. Female connector 978 is fixedly mounted in shaft 983, has a plurality of female sockets 979 for reception of pins 921 from handpiece 910, and has lower opening 978a for passage of drive member 923 therein. Key slot 983 is engaged with key 924 of the handle 910 to align or orient shaft assembly 970 to universal handpiece 910, and is passing blade 922b of shaft retention lock 922 therein. Spring 933 is biasing shaft retention lock 922 upwardly.

To complete installation of shaft assembly 970 into handpiece 910, shaft retention lock 922 must be depressed to bias drive blade 922b downwardly into alignment with actuator channel 977 of and interchangeable shaft assembly 970 and to move lock blades 922c of shaft retention lock 922 downwardly to allow passage of shaft assembly 910 through opening 922a. Shaft assembly 910 is shown fully inserted into shaft socket 920 which engages male pins 921 in handle 910 to shaft female sockets 979 of shaft assembly 970, and fully engages generator 902 to shaft assembly 970 through universal handle 910. Release of shaft retention lock 922 results in spring 933 biasing shaft retention lock 922 upwardly, engaging lock blades 922 with shaft retention slots 984 and securing shaft 970 to handle 910. Release of shaft retention lock 922 also releases drive member 923 by moving unlocking blade 922b upwardly enabling drive member spring 923a to bias tab 923b into locked engagement with slot 977a of actuator 977. When tab 923b is locked into slot 977a, proximal and distal movement of plunger 914 moves actuator channel 977 of shaft assembly 970 in a like manner to actuate and de-actuate end effector 975. Thus, universal handle 910 can be removeably engaged with any one of a number of shaft assemblies 970. If desired, electronics can be deleted from a shaft assembly 970 and universal handpiece 910 can operate a completely mechanical shaft assembly 970 with plunger 914.

The universal handle 910 preferably includes a circuit board 930 that can provide a thermocouple pass through, an EEPROM pass through, a LED pass through, a shaft indicator pass through, or any one of a number of other pass throughs for shaft electronics. Additionally, electrode switching circuits can be on the circuit board 930, as well as a mount for the activation switch. In addition, a shaft retention lock out can be included to lock out engagement of generator 902 unless a shaft assembly 970 is fully inserted and locked to handle 910. If desired, a generator activation switch 916 can be added to actuate generator 902 from handpiece 910, and, if desired, may not actuate until shaft retention lock out 990 is actuated by an inserted shaft assembly 970. A shaft retention lock out can be included to lock out generator 903 until shaft retention lock 922 is fully locked. Shaft connector 932 can be mounted to the circuit board 930 if desired or connected with cabling or flex circuitry. Actuation connector 928 and RF power connector 927 can also be attached to the circuit board 930 in universal handle 910. A battery and switch can be added to handpiece 910. The switch 993 locks out the battery to conserve power and can be engaged by removal of a battery lock tab or manual actuation of switch 993.

By way of example, a circuit board for a left clamp assembly or right clamp assembly may be provided for attachment to universal handle 910. Clamp shaft assemblies can be electrically identical but differ mechanically. Two pairs of opposed electrodes may be provided with connections to a connector. A thermocouple may also be provided and extends to the connector. Shaft electronics such as an EEPROM can contain information about the shaft assembly that enables generator 903 to recognize the shaft assembly and end effector and set generator operating parameters to match. A shaft present loop can also be provided to indicate to the universal handpiece 910 and generator 903 that an interchangeable shaft assembly is operably connected to handpiece 910.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

While preferred embodiments of the present invention have been shown and described herein, it will be obvious to those skilled in the art that such embodiments are provided by way of example only. Numerous variations, changes, and substitutions will now occur to those skilled in the art without departing from the invention. Accordingly, it is intended that the invention be limited only by the spirit and scope of the appended claims.

What is claimed is:

1. An electrosurgical system for coagulation of tissue comprising;
a) an electrosurgical generator capable of ablation and sensing;
b) an electrosurgical handpiece operably attached to said generator; and
c) a plurality of end effectors operably attachable and detachable to said electrosurgical handpiece, wherein when said end effector is attached to said handpiece, said end effector is recognized by one or more of said handpiece and said electrosurgical generator and appropriate operational parameters are selected for that end effector.

2. The electrosurgical device of claim 1 wherein said plurality of attachable and detachable end effectors is selected from the group of a left clamp, a right clamp, a dissector shaft and a band dispenser.

3. The electrosurgical device of claim 1 wherein each of said plurality of attachable and detachable end effectors has an electronic recognition key, said key containing information unique to that end effector, said information used by said electrosurgical generator to select generator operational parameters.

4. The electrosurgical device of claim 1 wherein said key is a programmed memory.

5. The electrosurgical device of claim 1 further comprising a connector, a key, a shaft alignment feature, and an actuator member operably coupled to said shaft, so that when connected, the shaft is locked to handpiece, a plurality of electrical connections are made, and shaft actuation mechanism is engaged.

6. The electrosurgical device of claim 1 wherein each of said plurality of attachable and detachable end effectors has at least one selected from the group of an electrode, a thermocouple, a light emitting diode and a fastener.

7. The electrosurgical device of claim I wherein said attachable and detachable end effector is a clamp.
